(19) United States
(12) Patent Application Publication
(10) Pub. No.: US 2014/0350447 A1
(43) Pub. Date: Nov. 27, 2014

(54) GEL-SIDE CUSHION

(71) Applicant: Hallufix AG, Munich (DE)

(72) Inventor: Manfred Brass, Munich (DE)

(73) Assignee: Hallufix AG, Munich (DE)

(21) Appl. No.: 14/284,477

(22) Filed: May 22, 2014

(30) Foreign Application Priority Data
May 24, 2013 (DE) 20 2013 004 834.4

(51) Int. Cl. A61F 5/01

(52) U.S. Cl. CPC A61F 5/0127
USPC 602/30

(57) ABSTRACT

An orthopedic device for correcting malpositions of toes comprises a flexible splint extending along the foot's inner side. A corrective force towards the inner side of the foot can be exerted on the big toe by the flexible splint for medializing the big toe. The corrective force is influenced by the spring stiffness of the flexible splint which is configured as a hinged flexible splint flexible in the flexion or extension direction of the toes and has a hinge mechanism that has a pivot axis that corresponds approximately to the hinge axis of the main big toe. The device has a first annular brace that surrounds the mid-foot on the outside and surrounds the flexible splint and a second annular brace which surrounds the big toe and the flexible splint. A gel cushion connects to that side of the second hinged splint facing towards the foot's inner side.

### CROSS-REFERENCE TO RELATED APPLICATION

Priority to German patent application number 20 201 3 004 834.4 filed on May 24, 2013, the entire disclosure of which is incorporated herein by reference, is claimed.

### FIELD OF DISCLOSURE

The present disclosure relates to an orthopedic device for correcting malpositioning of toes.

### BACKGROUND

A foot brace for treating malpositioning of the big toe, for example hallux valgus, is known from DE 100 34 354 A1. In the longitudinal direction of the foot, said foot brace has a stretchable receiving device for the big toe, a free end of which is connected to an annular brace that is arranged in the region of the mid-foot and surrounds the same so that a correcting force acts on the big toe in the direction of the anatomically correct position of the toe.

Experience has shown that patients wear such devices only reluctantly and unreliably because they are very thick and are found to be inconvenient in ordinary shoes, or even, when worn for long periods, to be tiresome or painful. The success of the treatment when using such a brace is therefore not assured.

From DE 1 881 215 U1, a bunion splint is known that acts as a bending spring that extends along the inner side of the foot and has a ring eye at the toe end that is used for receiving the big toe. At the opposite end, the bunion splint has a bend that can be placed around the heel. This enables to bring the big toe from an inwardly bent malposition of the toe into the normal position. This splint has significant disadvantages, e.g., those who wear them find them to be extremely uncomfortable in use so that it is only very reluctantly worn and consequently, the success of the treatment is not ensured.

A device for treating big toes is known from the German utility model DE 8 902 545.8 U1, said device having a stocking with a pouch that surrounds the big toe and having a splint that extends along the inner side of the foot and that is arranged in a pocket sewn onto the stocking. Such a device for treating big toes is intended for treatment during nighttime or when the patient sleeps. The disadvantage is that the freedom of movement of the splinted big toe in the direction of flexion/extension of the big toe is inhibited. Therefore, this device is not suited for long term treatment. Wearing this device inside a shoe is very uncomfortable for a patient and considerably restricts the freedom of movement.

Furthermore, spreading devices are known that are formed as a wedge and are disposed in the space between the big toe and the second toe so that the big toe is pushed towards the inner side of the foot. These devices have the disadvantage that in order to exert a force, they have to be supported by the adjacent toe and thus can cause or promote malpositioning of the adjacent toes. Object of present disclosure.

An orthopedic device for correcting malpositioning of toes is known from 102 40 121 B4, comprising a fastening provision (8a) in the region of the big toe, a fastening provision (8b) in the region of the mid-foot and a flexible splint (9) that extends between the fastening provisions (8a, 8b) along the inner side of the foot, wherein the flexible splint (9) is formed as a hinged splint element which is formed in a hinged manner in the direction of flexion/extension of the toe or the toes.

### GENERAL DESCRIPTION

It is an object of the present disclosure to provide a device by means of which valgus malpositions of toes, i.e., malpositions of one or more toes towards the outside of the foot can be treated and, moreover, the comfort when wearing the device is improved. Furthermore, wearing the device shall be comfortable, in particular without noticeable impairment when wearing it in everyday life. The success of treatment shall be improved with respect to the prior art.

An orthopedic device according to the present disclosure for correcting malpositions of toes comprises a flexible splint that extends along the inner side of the foot, wherein for medializing the big toe, a corrective force Fl towards the inner side of the foot can be exerted on the big toe by the flexible splint, wherein said corrective force is influenced by the spring stiffness of the flexible splint. In addition, the flexible splint is configured as a hinged flexible splint which is hinged (or articulated) in the direction of flexion or extension of the toes to be corrected and has a hinge mechanism that has a pivot axis that corresponds approximately to the hinge axis of the main big toe joint in the direction of flexion or extension, i.e., is aligned with the hinge axis. The flexible splint has a first hinged splint leg and a second hinged splint leg which are connected by means of the hinge mechanism to be pivotable about the pivot axis. Transmitting the corrective force Fl is ensured by annular braces made from a bendable, pliant, circularly tension-resistant material. In the region of the mid-foot, the device has a first annular brace that surrounds the mid-foot on the outside and surrounds the flexible splint. In the region of the free end of the big toe, the device has a second annular brace which circumferentially surrounds the big toe and the flexible splint. A gel cushion is connected to that side of the second hinged splint that faces towards the inner side of the foot.

Within the context of the present disclosure, the flexible splint is to be understood as a splint that has inherent material elasticity. The material elasticity effects an elastic reset force according to Hooke's law. The reset force affects the corrective force, wherein the corrective Fl is transmitted via the annular brace onto the big toe. The flexible splint can be deformed in particular in the horizontal direction so that the corrective force also acts in the horizontal direction. The hinge of the flexible splint preferably has slots, in particular at the hinge-side end of one of the at least two hinged splint legs. For example, these slots are curved on the inner side of the hinge, are arranged to be covered by the gel cushion and form an interrupted circle. The slots can be utilized for maintaining the hinge, for example.

The gel cushion is preferably made from a gel material that combines the three-dimensional deformation with the shape memory of a solid body. Also, it is preferably a nontoxic and stable polyurethane substance that is produced without using oils or plasticizers. In this way, skin irritation can be avoided. Moreover, the gel cushion is UV-resistant and waterproof as well as sweat-resistant. In addition, the heat conductivity of the gel cushion helps avoid overheating the foot, i.e., it has a cooling effect on the foot. The advantage of such a gel cushion is the gain in comfort when wearing the hinged splint.

The gel cushion provides three-dimensional deformation properties and a shape memory. Under the influence of pressure by the body, the cushion adapts to the shape of the foot by deforming along all three axes.

The gel cushion has elasto-mechanical properties resulting in improved damping behavior. By the cushion exactly adapting to any foot shape, it provides pressure relief since it distributes the pressure forces uniformly over a larger foot surface. Thus, load peaks are absorbed. In this way, muscles, joints, tendons and ligaments are protected.

The gel cushion is preferably coated on one side, at least partially, with a textile material. For example, the gel material can be applied in the heated state onto the textile and can subsequently be bonded to the textile by cooling. Alternatively, the textile can also be glued onto the gel cushion. The textile offers advantages when fastening the gel cushion on the splint. The gel cushion can preferably be connected to the leg of the splint via the textile by means of a Velcro fastener or a hook and loop fastener.

### BRIEF DESCRIPTION OF THE DRAWINGS

The present disclosure is exemplary explained below in greater detail with reference to the drawings. In the figures:

FIG. 1 schematically shows a bottom view onto a device according to the present disclosure;

FIG. 2 schematically shows a device according to the present disclosure in a view onto the inner side of the foot;

FIG. 3 schematically shows a hinged splint of the device according to the present disclosure in a perspective detailed view;

FIG. 4 schematically shows the hinged splint from FIG. 3 in a top view;

FIG. 5 shows a top view onto a gel cushion according to the present disclosure, and

FIG. 6 shows a side view of a gel cushion according to the present disclosure.

### DETAILED DESCRIPTION

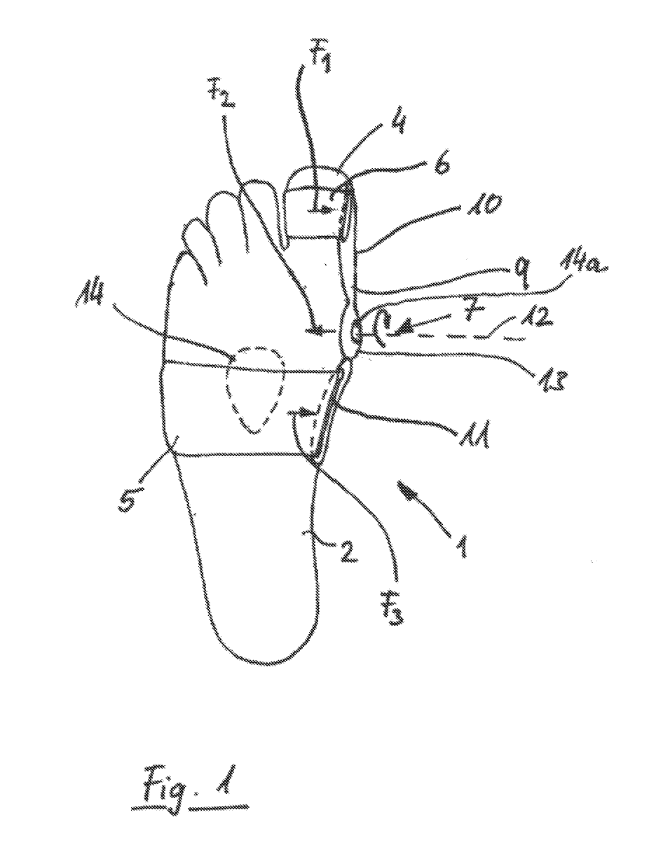

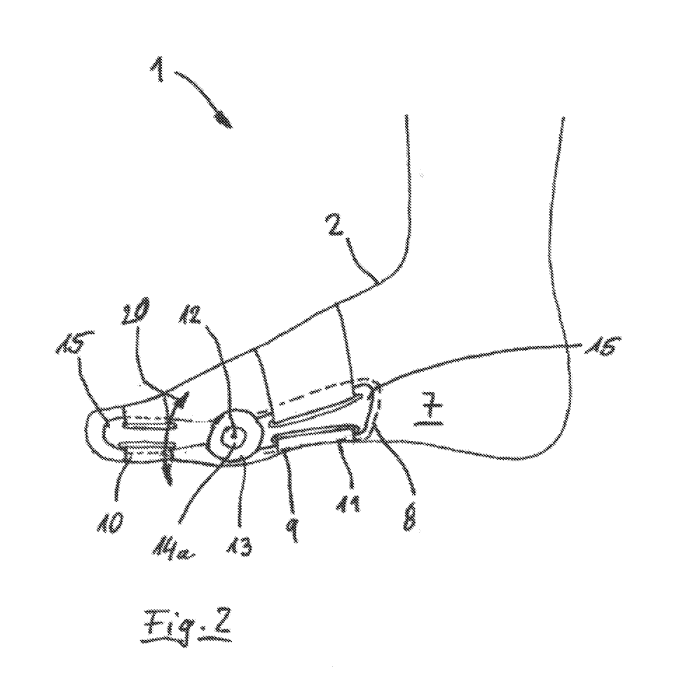

A first embodiment of the device 1 according to the present disclosure (FIG. 1) has a receptacle for a big toe, e.g., a big toe pouch 4. The big toe pouch 4 circumferentially surrounds the big toe preferably completely and is open in the region of the free toe end.

In the region of the mid-foot, the device 1 according to the present disclosure has a first annular brace 5 which surrounds the mid-foot preferably completely.

In the region of the free end of the big toe, the big toe pouch 4 is formed as a second annular brace 6 which circumferentially surrounds the big toe preferably completely. The annular braces 5 and 6 are preferably made from a bendable, pliant, circularly tension-resistant material, i.e., a material that is tension-resistant in the circumferential direction, for example, a fabric tape or a tension-resistant adhesive tape.

The longitudinal side that faces towards the adjacent foot is defined as the inner side of the foot 7. The outer side of the foot is the longitudinal side of the foot that is located opposite from the inner side.

The flexible split 9 extends along the inner side of the foot 7. The flexible splint 9 is configured as a hinged flexible splint and has a first hinged splint leg 10 and a second hinged splint leg 11, which are hingedly connected to one another by means of a hinge mechanism 13 to be pivotable about a pivot axis 12. The hinge mechanism 13 is arranged with respect to the foot in such a manner that the pivot axis 12 corresponds approximately to the hinge axis of the main big toe joint in the direction of flexion or extension 20, i.e., in the natural bending direction, thus the dorsal and plantar bending direction. The first hinged splint leg 10 extends starting from the hinge mechanism 13. The hinge mechanism 13 is positioned on patients approximately in the region of a swelling (pseudoexostosis) on the inner side of the foot which is typical for hallux-valgus malpositioning and which often projects on the inner side of the foot beyond the proper contour of the foot. Thus, when wearing a device 1 according to the present disclosure, a force F1, which acts in the mediolateral direction towards the inner side of the foot, is exerted in the region of the big toe onto the big toe. In the region of the main toe joint, a force F2 is exerted via the swelling in the opposite direction. A support force F3 resulting from the forces F1 and F2 is absorbed by the first annular brace 5.

Advantageously, for retrocapital support of the mid-foot, a splayfoot pad 14 is inserted preferably in a removable manner in the annular brace 5 in the region of the foot sole behind the main joints of the toes. Said splayfoot pad supportingly raises the transverse arch, which has another positive influence on the correction of toe malpositioning. However, a configuration without a pad is also possible.

In the first embodiment according to FIG. 1, the circularly tension-resistant annular braces 5, 6 ensure sufficient force transmission. In the simplest conceivable embodiment, the device 1 according to the present disclosure may consist merely of the annular braces 5, 6 and the flexible splint 9 so that the flexible splint rests directly against the foot skin of the patient. However, this simplest embodiment of the device 1 according to the present disclosure provides relatively low wearing comfort, but it implements the inventive idea. This wearing comfort is noticeably improved by the gel cushion according to the present disclosure located between the foot and the second hinged splint leg 11.

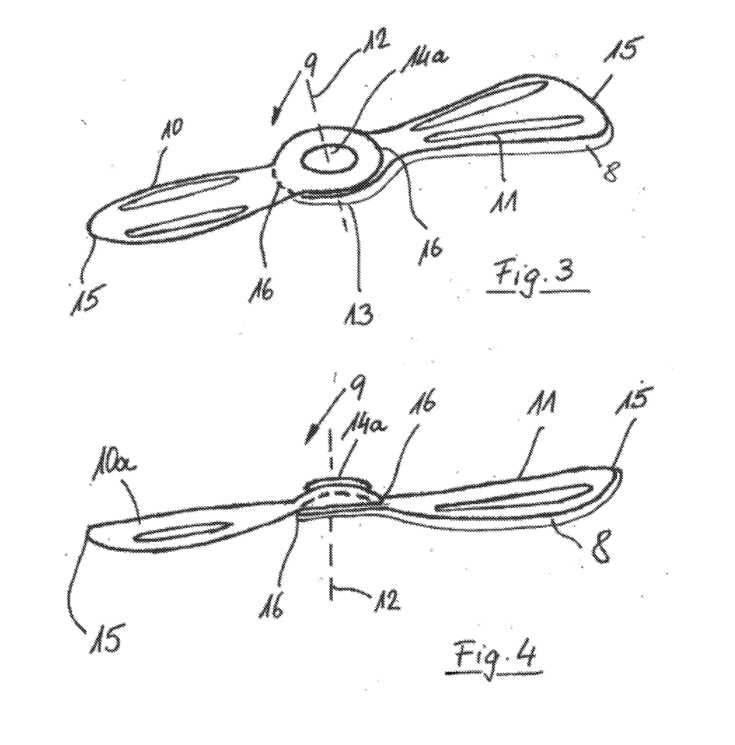

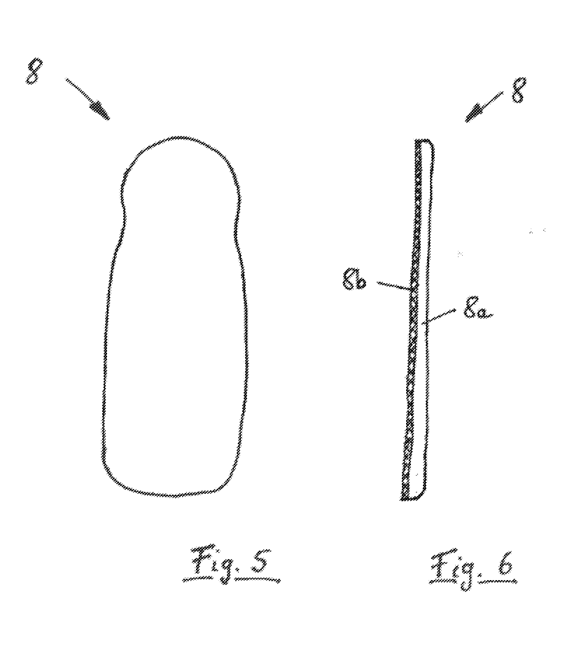

In the FIGS. 3 and 4, the gel cushion 8 is illustrated as it is arranged on the second hinged splint leg 11. In the worn state, the gel cushion 8 is located between the second hinged splint leg 11 and the patient's foot (not shown). The gel cushion 8 is also arranged between the hinge mechanism 13 and the patient's foot. The FIGS. 5 and 6 show the gel cushion. The side view in FIG. 6 shows the structure of the gel cushion from a gel material 8a and a textile material 8b. The two layers or materials 8a, 8b are bonded by applying the heated gel onto the textile and subsequent cooling of the gel. The textile material 8b is connected to the second hinged splint leg 11 via a hook and loop fastener. In the worn state of the hinged splint 9, the gel material rests against the patient's skin or foot.

The hinged mechanism 13 is substantially constructed from three pieces including the first hinged splint leg 10, the second hinged splint leg 11 and a connecting device 14a for the hinged splint legs, in particular a tubular rivet. The hinged splint legs 10, 11 each have a free end 15 and an end 16 at the hinge. The ends 16 at the hinge have approximately the shape of a universal ball joint and are formed to correspond to one another in such a manner that each of the hinge-side ends 16 of the hinged splint legs 10, 11 can be engaged with one another in a form-fitting manner. The hinge-side ends 16 in the shape of universal ball joints, which are connected by means of the tubular rivet 14a, form the hinge mechanism 13 which, due to the of the hinge-side ends 16 shaped as a universal ball joint, has high stability in the mediolateral direction.

Furthermore the bulge of the hinge mechanism 13, as a combined effect, has the advantage that in this way, individual adaptation of the hinge mechanism 13 to the patient's foot contour in the region of the main big toe joint is possible in a simple manner. This is particularly advantageous since in the case of an existing hallux-valgus malposition, ball-shaped swellings (pseudoexostosis) are often found on the foot in the region of the main big toe joint. The shape, depth, size and diameter of the universal ball joint are individually adapted to the patient's foot in a particularly advantageous manner.

The hinged splint legs 10, 11 which extend away from the hinge mechanism 13, are advantageously formed such that they match the contour of the foot in terms of the cross-section or the entire spatial shape. In addition to the spatial shapes in the form of a universal ball joint, any kind of rotationally symmetric spatial shapes that correspond to one another, in particular truncated cone-shaped spatial shapes or spatial shapes having a parabolic cross-section, are suitable for the formation of the hinge-side ends 16 of the hinged splint legs 10, 11.

Metal or plastics are particularly suitable as materials for the flexible splint 9 or the hinged splint legs 10, 11, which preferably are formed as elastic, thin and plate-shaped elements, wherein a thin carbon-fiber-reinforced plate was found to be particularly advantageous because it can easily be formed under the influence of heat and provides high spring force with a low material thickness after cooling. The stiffness of the flexible splint 9 in the mediolateral direction is further increased by a non-planar cross-sectional shape of the legs 10, 11. Thus, with a device according to the present disclosure having a flexible splint 9, one the one hand, high wearing comfort can be achieved because the flexible splint 9 can be individually adapted the patient's foot, and, on the other hand, increased spring stiffness in the mediolateral direction is achieved so that as a substantial result of this, a surprisingly great treatment success can be implemented.

A device according to the present disclosure also ensures that only a very thin material layer is to be applied on the inner side of the foot or the big toe to be corrected so that the device 1 according to the present disclosure can be worn without noteworthy difficulties in normal shoes. Also, the freedom of movement of the big toe is not restricted since the device 1 according to the present disclosure makes it possible for the toe to move in the natural direction of flexion or extension 20 or, respectively, the freedom of movement is not restricted in this direction. This device 1 therefore is particularly suitable for long-term treatments by day and by night since the patient does not experience any noteworthy hindrance caused by this device 1.

With regard to their cross-sectional spatial shape, the hinged splint legs 10, 11 are non-planar, i.e., for example, curved with a constant thickness formed from a sheet material, wherein the shape is adapted to the patient's individual foot contour. Furthermore, it is of course also possible for the hinged splint legs 10, 11 to have a lenticular shape in cross-section, in particular to have a lenticular shape in such a manner that the material thickness decreases towards the edges of the hinged splint legs 10, 11 so that the geometrical adaptation of the flexible splint 9 to the patient's foot is individually further improved.

According to another embodiment (not shown), the orthopedic device 1 according to the present disclosure for correcting malpositions of a plurality of adjacent toes is improved in that, e.g., tensioning means extend from the annular brace 6 around the big toe and act on one or more adjacent toes which, for example, are likewise surrounded by an annular toe brace. The corrective force of the flexible splint 9 in the mediolateral direction thus can be transmitted in a simple manner from the big toe to adjacent toes. Through appropriate selection of the material thickness for the flexible splint legs 10, 11 and through appropriate selection of the peripheral cut of these hinged splint legs 10, 11 and through simple adaptability of the spring preload of the flexible splint 9 in the mediolateral direction, the spring stiffness and thus the force F1 correcting the toe position can be influenced in a simple manner.

Individual adaptation of the corrective force F1 to specific needs of the patient is therefore possible with simple means that are available in any orthopedic workshop such as, e.g., adapting the circumferential contour of the legs 10, 11, or changing the cross-sectional contour of the legs 10, 11 and/or the hinge mechanism 13.

We claim:

1. An orthopedic device for correcting malpositions of toes, comprising a flexible splint which extends along the inner side of the foot, wherein for medializing the big toe, a corrective force F1 can be exerted by the flexible splint onto the big toe in the direction towards the inner side of the foot, which corrective force is influenced by the spring stiffness of the flexible splint, and wherein the following features are provided:
a) the flexible splint is configured as a hinged flexible splint which is hinged in the direction of flexion or extension of the toes to be corrected and has a hinge mechanism that has a pivot axis that corresponds approximately to the hinge axis of the main big toe joint of the in the direction of flexion or extension;
b) the flexible splint has a first hinged splint leg and a second hinged splint leg (11) which are connected by means of the hinge mechanism to be pivotable about the pivot axis;
c) transmitting the corrective force F1 is ensured by annular braces made from a bendable, pliant, circularly tension-resistant material;
d) in the region of the mid-foot, the device has a first annular brace that surrounds the mid-foot on the outside and surrounds the flexible splint;
e) in the region of the free end of the big toe, the device has a second annular brace which circumferentially surrounds the big toe and the flexible splint,
characterized in that
f) a gel cushion is connected to that side of the second hinged splint that faces towards the inner side of the foot.

2. The device according to claim 1, wherein the hinged splint legs are lenticularly shaped in cross-section.

3. The device according to claim 1, wherein the gel cushion (8) is connected to the second hinged splint leg in a detachable or non-detachable manner.

4. The device according to claim 1, wherein at least a portion of the surface of the gel cushion is coated with a textile .

5. The device according to claim 4, wherein the gel cushion is detachably connected to the second hinged splint leg via a hook and loop fastener connection.

6. The device according to claim 1, wherein a splayfoot pad is inserted in the region of the foot sole behind the main joints of the toes for retrocapital support of the mid-foot.

7. The device according to claim 1, wherein the pivot axis of the hinged mechanism is constructed as a connecting device for the hinged splint legs using a tubular rivet.

8. The device according to claim 1, wherein the hinged splint legs each have a free end and an end at the hinge, wherein the hinge-side ends of the hinged splint legs have approximately the shape of a universal ball joint and are formed to correspond to one another in such a manner that each of the hinge-side ends of the hinged splint legs can be engaged with one another in a form-fitting manner.

9. The device according to claim 1, wherein the hinged splint legs and the hinge mechanism are formed such that their spatial shape is adapted to the patient's foot contour.

10. The device according to claim 1, wherein the hinge-side ends of the hinged splint legs have spatial shapes that are rotationally symmetric about the axis and that correspond to one another.

11. The device according to claim 1, wherein the hinged splint legs are made from metal or plastics, in particular from a thin carbon-fiber-reinforced plate.

12. The device according to claim 1, wherein at least one of the two hinged splint legs has slots at the hinge-side end .

13. The device according to claim 1, wherein means are available that allow the corrective force F1 to also act on one or more adjacent toes.

14. The device according to claim 12, wherein said means are tension means that interconnect toe receptacles for a plurality of toes of a foot.
